(19) United States
(12) Patent Application Publication
(10) Pub. No.: US 2022/0032285 A1
(43) Pub. Date: Feb. 3, 2022

(54) Filter Sampling Bag

(71) Applicant: Interscience, St Nom la Breteche (FR)

(72) Inventor: Emmanuel Jalenques, St Nom la Breteche (FR)

(21) Appl. No.: 17/372,822

(22) Filed: Jul. 12, 2021

(30) Foreign Application Priority Data
Jul. 28, 2020 (FR) 20 07977

(51) Int. Cl. B01L 3/00

(52) U.S. Cl. CPC B01L 3/505; B01L 2200/12; B01L 2300/02; B01L 2300/0858; B01L 2300/0851; B01L 2300/0681

(57) ABSTRACT

A filter sampling bag for use in a mixer for the preparation of samples for carrying out analyses and/or microbiological tests, the sampling bag formed from a plastic and having a bottom, side walls, and a mouth opening. The sampling bag is compartmentalized by a filtering partition delimiting a receiving chamber for a sample and a sampling chamber to permit a taking of samples. The sampling bag is marked with a visual indicator of a correct, predetermined closing position of the sampling bag after mixing. A sampling set or kit comprising a mixer and a filter sampling bag as disclosed, and a method of manufacturing such a filter sampling bag for use in a mixer for the preparation of samples to permit analyses and/or microbiological tests.

### RELATED APPLICATION

This application claims priority to French Application No. 2007977, filed Jul. 28, 2020.

### FIELD OF THE INVENTION

The present invention relates to the field of sample collection and preparation for analysis, and in particular to the preparation and collection of a filtrate from a solid sample. Still more particularly, embodiments of the present invention comprise a filter collection bag, such as a flat bag of transparent plastic material, compartmentalized by a filter partition to delimit a collection chamber for taking samples that can be used for the preparation of a sample filtrate from a potentially contaminating solid or liquid sample. A solid sample could be fibrous, inhomogeneous, and insoluble. In such cases, a liquid, such as a physiological or culture medium, can be placed in the bag. The sample, when placed in the compartment, is in contact with the liquid. The amount of liquid used is usually one-tenth the weight of the solid sample and usually represents only a small volume at the bottom of the bag. The filtrate can be viscous or pasty. The analysis of the filtrate can, in particular, be microbiological.

### BACKGROUND OF THE INVENTION

Filter collection bags are used in a variety of circumstances. For instance, filter bags may be used when a sample to be analyzed is solid and poorly soluble, where a sample is difficult to handle, where a sample is potentially pathogenic, and, additionally or alternatively, where the sample taken “on site” must not be contaminated. Typically, microbiological analysis processes require the dilution of solid or liquid samples to be analyzed to obtain a liquid form.

Most filter collection bags have two compartments separated by a filter. One compartment is intended to receive a solid sample while the other compartment forms a collection chamber intended to receive the filtrate. According to one possibility, the filter may be what can be referred to as a full-page type that separates the bag into two identical compartments. One compartment is intended to receive the sample and the diluent, and the other compartment is intended to collect the filtered mixture using a pipette. According to another possibility, the filter can be of a lateral type with a lateral chimney formed to divide the bag into two compartments. One compartment, which can be a larger compartment of the two, receives the sample and the diluent.

Sampling bags typically have dimensions of 95×180 mm for a maximum mixing volume of 100 mL, 190×200 mm for a maximum mixing volume of 400 mL, 250×380 mm for a maximum mixing volume of 2000 mL, and 380×600 mm for a maximum mixing volume of 3750 mL.

With each such embodiment, the filter collection bag with the sample and the diluents therein can be inserted into a mixer to aid in suspension of the sample and/or solubilization of solid sample objects in the liquid. After grinding, the compartment intended to receive the filtrate forms a sampling chamber that makes it possible to take the suspension obtained after filtration. All or part of the filtrate is then taken for analysis.

To prevent capillary rise and leakage from the top of the bag during mixing, the bag must be kept closed at a predetermined height. This can be done, by way of example and not limitation, by pinching the bag in the mixer door. The present inventor has devised of mixers with special closures, such as the specialized closure for mixers sold by Interscience SARL of Saint-Nom La Breteche, France under the trademark Q-Tight, to eliminate any risk of leakage from the top of the sampling bag during mixing. A lever ensures that the mixer door is locked in a closed position with the door then maintaining the bag in a closed configuration.

Following mixing, it may be necessary to close the bag temporarily, such as to place it on a bag rack for storage. To that end, after mixing, the user is required to make a fold across the width of the bag and then to install a bag clasp or any other pinching device on the fold.

The products analyzed are often fatty and facilitate the circulation of the liquid in the bag during mixing with the walls of the bag becoming greasy. As such, the walls of the bag often do not adhere to each other such that the liquid can rise to an excessive height within the bag during mixing.

Thus, under the effect of mixing, the liquid contained in the bag rises and permeates the filter, which acts like a blotter and absorbs the contaminated medium. During this step, if the closure is too low and is below the pinch area of the mixer door maintaining pressure during mixing, the medium contained in the bag is frequently spilled from the bag. That discharge may be more or less rapid, and the user may not immediately notice their error in making the bend. In addition, the volume of liquid increases under the effect of heat and bacterial growth to be analyzed during an enrichment and incubation step. With prior art bags, the potentially contaminating medium contained in the bag thus frequently spills outside of the bag.

The foregoing problems have existed in the art for some time, and there is a recognized need File Reference: INTER-MUS for a filter sampling bag that presents a viable solution thereto.

### SUMMARY OF THE INVENTION

According to a first aspect of the invention, there is provided a filter sampling bag that is preferably flat, preferably transparent with a bottom, side walls, and a mouth opening. The bag is formed from a plastic material and is compartmentalized using a filtering partition delimiting a chamber for receiving a sample and a sampling chamber permitting a taking of samples. The bag has a visual indicator of a correct closing position of the bag. With such an indicator, the user knows where to make a fold in the bag to close the bag while avoiding spillage outside of the bag of the medium contained in the bag.

According to one possibility, the visual indicator is disposed on the bag at a distance from the opening substantially equal to one third of the height of the bag. Preferably, the distance is equal to one third of the height of the bag plus or minus five percent of the height of the bag (between 28% and 38% of the height of the bag).

According to a second aspect of the invention, there is provided a sampling assembly comprising a plastic sampling bag according to the first aspect of the invention and a mixer for preparing samples with a view to permitting the performance of microbiological analyses and/or tests.

According to a third aspect of the invention, there is provided a sampling kit comprising a mixer for preparing samples with a view to performing microbiological analyses and/or tests and a plastic sampling bag according to the first aspect of the invention.

According to a fourth aspect of the invention, there is provided a method of manufacturing a sampling bag intended to be used in a mixer for the preparation of samples with a view to carrying out microbiological analyses and/or tests, the sampling bag having a bottom, side walls, and an opening, the sampling bag being compartmentalized by a filtering partition delimiting a chamber for receiving a sample and a sampling chamber serving to take samples, the method comprising a step of marking the sampling bag with a visual indicator of a correct, predetermined closing position of the bag after mixing.

The method according to the invention may further include, prior to the marking step, a step of determining the position of the visual indicator from the closed position of the bag in the mixer.

One will appreciate that the foregoing discussion broadly outlines the more important goals and features of the invention to enable a better understanding of the detailed description that follows and to instill a better appreciation of the inventor's contribution to the art. Before any particular embodiment or aspect thereof is explained in detail, it must be made clear that the following details of construction and illustrations of inventive concepts are mere examples of the many possible manifestations of the invention.

### BRIEF DESCRIPTION OF THE DRAWINGS

Other advantages and features of the invention will become apparent on reading the detailed description of implementations and embodiments, which are in no way limiting, with regard to the accompanying drawings, in which:

FIG. 1 is a schematic depiction of an open sampling bag according to the prior art prior to a mixing step;

FIG. 2 is a schematic depiction of the prior art sampling bag of FIG. 1 pinched along a pinching line in the mixing step;

FIG. 3 is a schematic depiction of the prior art sampling bag of FIG. 2, unpinned, subsequent to the mixing step;

FIG. 4 is a schematic depiction of the prior art sampling bag of FIG. 3 closed after closing along a fold line arranged under the clamping line subsequent to the step shown in FIG. 3;

FIG. 5 is a schematic depiction of a sampling bag according to the present invention subsequent to a mixing step;

FIG. 6 is a schematic depiction of a collection bag according to the invention, closed after closing along a fold line, subsequent to an opening step shown in FIG. 5; and

FIG. 7 is a schematic depiction of an alternative embodiment of a sampling bag according to the present invention.

### DETAILED DESCRIPTION OF PREFERRED EMBODIMENTS

The sampling bag disclosed herein is subject to varied embodiments, each within the scope of the invention. However, to ensure that one skilled in the art will be able to understand and, in appropriate cases, practice the present invention, certain preferred embodiments of the broader invention revealed herein are described below. The embodiments shown and described herein are not limiting. Variants of the disclosed embodiments may incorporate only a selection of characteristics described, potentially isolated from other described characteristics, particularly where the selection of characteristics is sufficient to confer a technical advantage or to differentiate from the state of the prior art. Sampling bags within the scope of the invention may thus incorporate one or more characteristics, perhaps functional without being limited relative to particular structural details or with only part of the structural details, where sufficient to confer a technical advantage or to differentiate the invention from the state of the prior art.

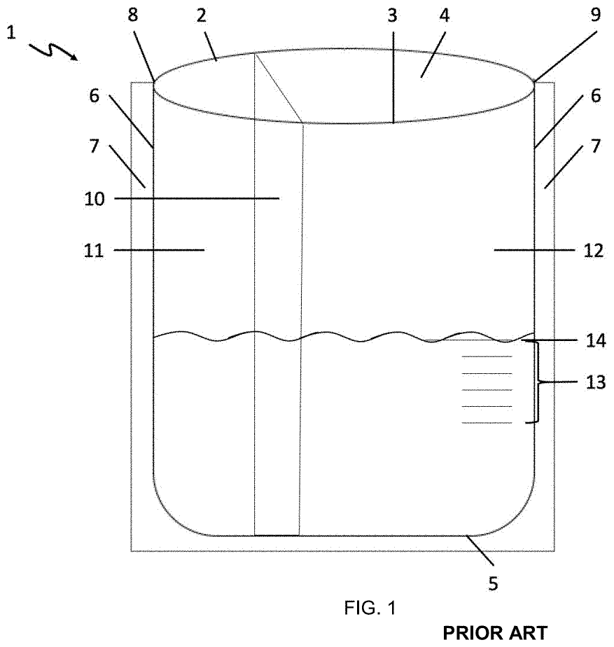

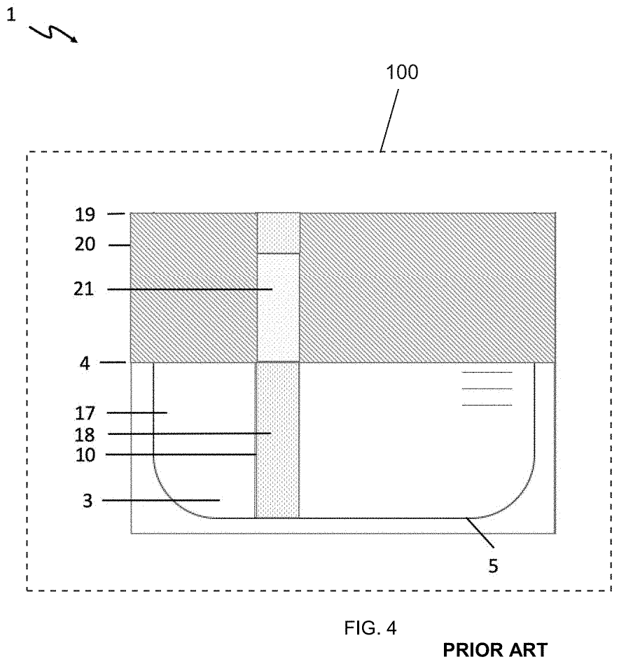

With further reference to the drawings, where like reference numerals are applied to like elements in the several figures, FIG. 1 illustrates a polyethylene collection bag 1 according to the prior art intended for use in a mixer, schematically illustrated and indicated at 100 in FIG. 4, for preparing samples for performing analyses and/or microbiological tests. The collection bag 1 may alternatively be referred to as a filter sampling bag 1. Samples can be solid and/or liquid. The collection bag 1 has two walls 2, 3 extending from top to bottom between a mouth opening 4 and a bottom 5 and extending, laterally, between two inner side edges 6.

According to embodiments presented, the lateral inner edges 6 are mainly parallel to each other. The sampling bag 1 is, in certain practices, manufactured by thermoforming two sheets of polyethylene by forming a fold 7 connecting two lateral ends 8, 9 of the opening 4, and extending along a first lateral inner edge 6, then along bottom 5 of the bag 1, and then along a second inner side edge 6 of the bag 1.

The bag 1 is compartmentalized by a filter partition 10 to define a first chamber 11 for receiving a sample and a second, sampling chamber 12 for permitting a taking of samples.

The filtering partition 10 can, for instance, be made of polypropylene with a cut-off threshold, such as of approximately 280 microns. The wall 3 can have a graduation 13. The graduation 13 can have a maximum graduation level 14 to be observed for correct use of the bag 1 in the mixer 100.

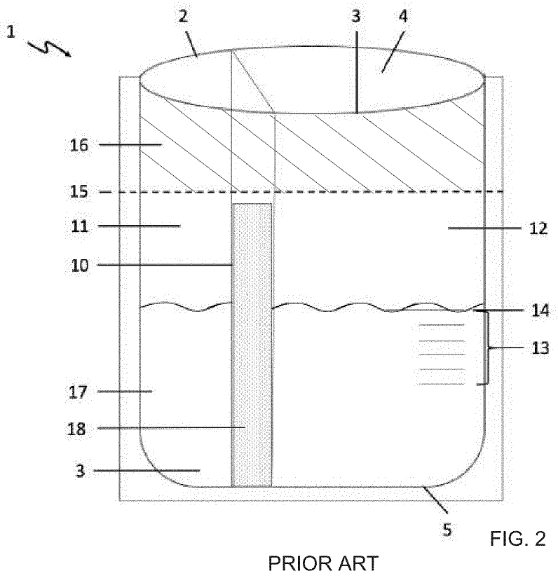

Referring to FIG. 2, when the prior art sampling bag 1 is introduced into the mixer 100, it is clamped along a clamping or pinch line, which is here drawn in dotted lines and indicated at 15. The clamping along the pinch line 15 is designed to make the sampling bag 1 liquid-tight during mixing.

Of course, air can escape from the sampling bag 1 past the pinch line 15, such as during mixing. The clamping line 15 can be considered to divide the bag 1 into an upper part 16 to the side of the mouth opening 4 of the bag 1 and a lower part 17 of the bag 1 to the side of the bottom 5 of the bag 1.

In FIG. 2, the upper part 16 is shown with a filling pattern with diagonal stripes extending from an upper left part to a lower right part. When the sampling bag 1 thus made liquid-tight by squeezing is stirred in the mixer 100, the sample is suspended in its liquid or optionally in an added diluent.

Referring again to FIG. 2, there is shown a soiled surface 18, which may alternatively be referred to as an impregnated surface 18. The soiled surface 18 is represented by a striped fill pattern with alternating vertical lines. In the diagram, the top level of the impregnated surface 18 is drawn at a level slightly below the pinch line 15, although it is noted that the the top level of the impregnated surface 18 could well be at the same level as the pinch line 15. Although the impregnated surface 18 is shown only over the width of the filter partition 10, the width of the soiled surface 18 extends over the entire bag 1, the liquid having circulated in the lower part 17 under the effect of the kneading of the mixer 100.

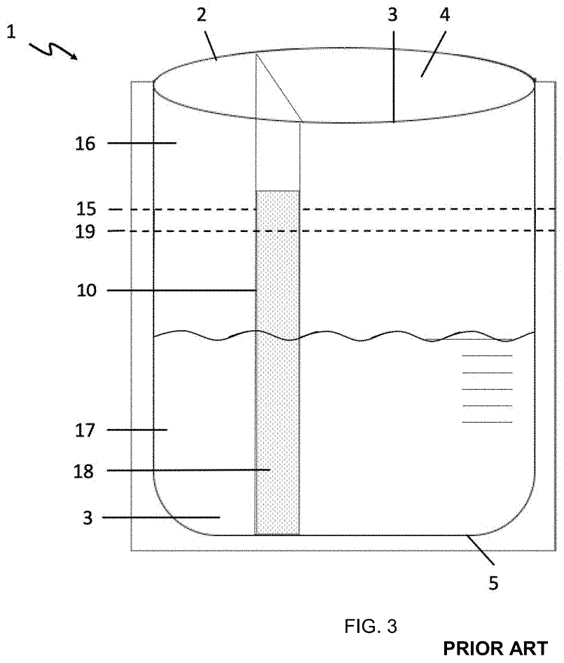

FIG. 3 schematically illustrates the state of impregnation of a prior art sampling bag 1 after it has passed through and been removed from the mixer 100. The sampling bag 1 is then no longer pinched along the pinch line 15.

The filtering partition 10 acts as a blotter. The suspension present at the level of the soiled surface 18 can rise by capillary action along the filtering partition 10, potentially invisibly or imperceptibly. The high level of the soiled surface 18 containing a potentially contaminating liquid can be found, again by capillary action, slightly above the clamping line 15.

With reference to FIGS. 3 and 4, it will be understood that closing the sampling bag 1 at a fold line 19 located below the top level of the pinch line 15 will result in a spill of potentially contaminating material outside of the prior art sampling bag 1 as is illustrated in FIG. 4.

In the example shown in FIG. 4, an upper part of the sampling bag 1 is folded over the wall 3 along fold line 19. According to another possibility and with reference to FIG. 3, the upper part of the sampling bag 1 could be folded over the wall 2.

When folded along the fold line 19, the upper part 20 of the filter wall 10 at the fold line 19 is folded down and contains any portion of the liquid contained in the sampling bag 1 in the filter wall 10 above the filter wall folding line 19. Under the effect of gravity, this part of contaminating liquid flows via the not-yet-impregnated part 21 of the filter partition 10 until it spills out of the bag 1 through the mouth opening 4. The flow of liquid creates a depression in the filtering partition 10 such that a siphon can be started. The flow will continue until the contents of the sampling bag 1 are almost completely emptied.

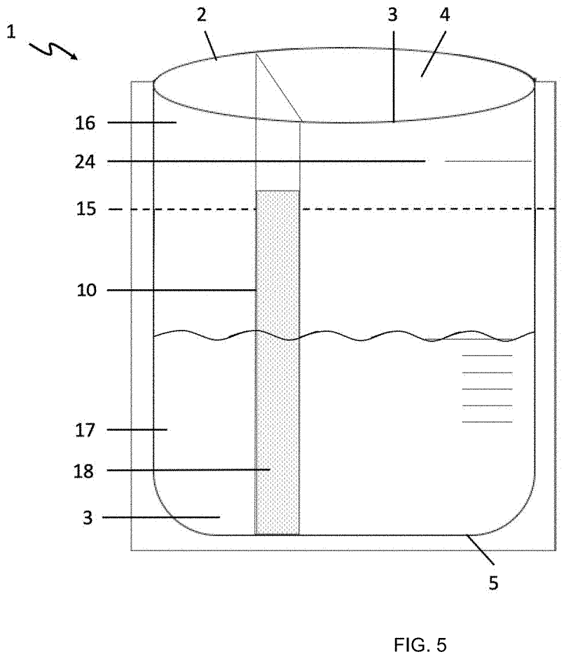

A sampling bag according to the present invention is indicated at 1 in FIG. 5. To alleviate the risk of the medium contained in the sampling bag 1 spilling outside the bag 1, the bag 1 according to the invention is marked with a visual indicator 24 of a correct closing position of the bag 1 after mixing.

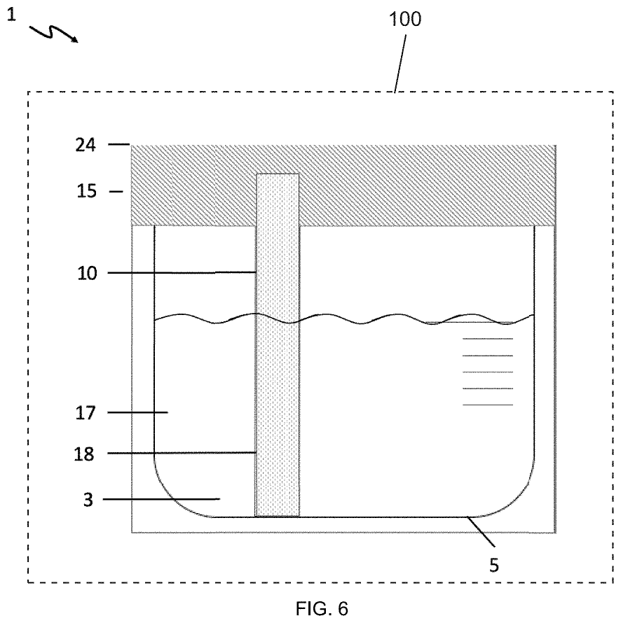

When the bag 1 according to the invention is closed along a fold line at the level of the visual indicator 24 as shown in FIG. 6, there is no risk of contamination beyond the closure of the bag 1. Thus, the bag 1 is marked with a visual indicator 24 of a correct, predetermined closing position of the bag 1 after mixing. As referenced herein, the correct closure indicator 24 is an indicator allowing closure of the sampling bag 1 to prevent potentially contaminating capillary leaks, rising contaminants, and upwelling and subsequent spillage of those contaminants outside the sampling bag 1. The sampling bag 1 is thus marked with a visual indicator 24 of a predetermined correct closing position of the sampling bag 1 after mixing thereby to avoid capillary leaks and rising contaminants from being poured or spilled from the sampling bag 1.

Pursuant to embodiments of the invention, the visual indicator 24 establishes a correct closure visual indicator that allows closure of the bag 1 in a manner that prevents potentially contaminating capillary leaks rising and then spilling outside the bag 1. The visual indicator 24 can take a variety of forms within the scope of the invention. In certain, non-limiting embodiments, the visual indicator 24 can comprise a distinct line that traverses laterally across either or both walls 2 and 3 as shown in FIG. 5.

As disclosed herein, the line position of the visual indicator 24 can be determined from knowledge of the bag closing line in the mixer. For example, the visual indicator 24 can be placed between the bag clamping line 15 and the mouth opening 4, such as in the middle between the bag clamping line 15 and the mouth opening 4. It is also possible in certain embodiments to place the visual indicator 24 at a distance from the opening 4 located at one third of the height of the sampling bag 1, plus or minus 5% of the height.

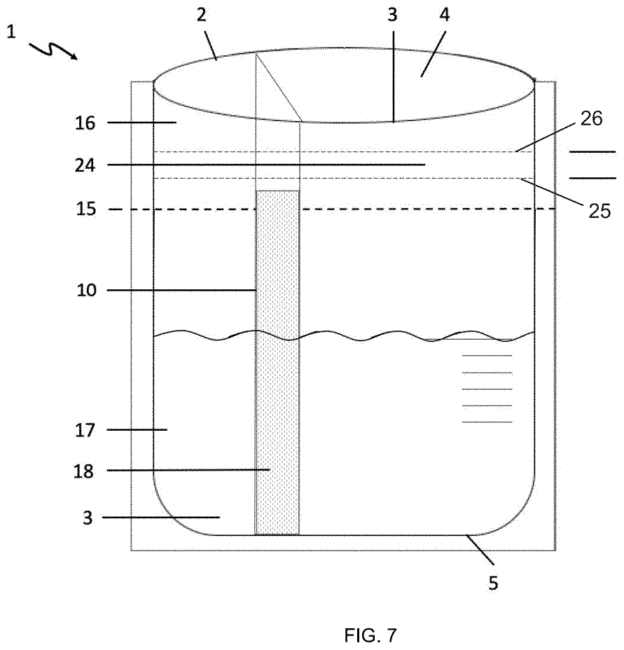

Of course, other types of visual indicators 24 can be provided. For example, the visual indicator 24 may be formed with two distinct lines. In particular, the visual indicator 24 can have a low line 25 and a high line 26 that together form a visual indicator 24 of the fold position as shown in FIG. 7. It is thus confirmed by the visual indicator 24 not only that the fold is not too low but also that it is not too high.

There is also proposed according to the invention a collection assembly comprising a mixer 100 for preparing samples with a view to performing analyzes and/or microbiological tests and a plastic collection bag 1 as has just been described. The folding height in the mixer 100 is then perfectly known.

Finally, there is proposed a method of manufacturing a sampling bag 1 intended to be used in a mixer 100 for the preparation of samples with a view to performing analyses and/or microbiological tests having a bottom 5, side walls 2, 3, and an opening 4, the sampling bag 1 being compartmentalized by a filtering partition 10 delimiting a first chamber 11 for receiving a sample and a second, sampling chamber 12 serving to take samples, the method comprising a step of marking the sampling bag 1 so formed with a visual indicator 24 of a correct, predetermined closing position of the sampling bag 1 after mixing.

Advantageously, the method further comprises, prior to the marking step, a step of determining the position of the visual indicator 24 from the closed position of the sampling bag 1 in the mixer 100.

Of course, the invention is not limited to the examples which have just been described. Numerous modifications can be made to these examples without departing from the scope of the invention. In addition, the different characteristics, shapes, variants and embodiments of the invention can be associated with each other in various combinations as long as they are not incompatible with or exclusive of each other.

When introducing elements of the present invention or embodiments thereof, the articles “a,” “an,” and “the” are intended to mean that there are one or more of the elements. The terms “comprising,” “including,” and “having” are intended to be inclusive such that there may be additional elements other than the listed elements. As used herein, terms such as “example” or “exemplary” are not intended to imply a superlative example. Rather, “exemplary” refers to an embodiment that is one of many possible embodiments.

With certain details and embodiments of the present invention for a sampling bag, sampling kit, sampling set, collection assembly, and method of manufacturing a sampling bag disclosed, it will be appreciated by one skilled in the art that numerous changes and additions could be made thereto without deviating from the spirit or scope of the invention. This is particularly true when one bears in mind that the presently preferred embodiments merely exemplify the broader invention revealed herein. Accordingly, it will be clear that those with major features of the invention in mind could craft embodiments that incorporate those major features while not incorporating all of the features included in the preferred embodiments.

Therefore, the following claims shall define the scope of protection to be afforded to the invention. Those claims shall be deemed to include equivalent constructions insofar as they do not depart from the spirit and scope of the invention. It must be further noted that a plurality of the following claims may express, or be interpreted to express, certain elements as means for performing a specific function, at times without the recital of structure or material. As the law demands, any such claims shall be construed to cover not only the corresponding structure and material expressly described in this specification but also all legally-cognizable equivalents thereof.

What is claimed as deserving the protection of Letters Patent:

1. A filter sampling bag to be used in a mixer for the preparation of samples to permit analyses and/or microbiological tests, wherein the sampling bag is formed of plastic material, wherein the sampling bag has a bottom, side walls, a mouth opening, and a height, wherein the sampling bag is compartmentalized by a filtering partition delimiting a receiving chamber for a sample and a sampling chamber for permitting a taking of samples, and wherein the sampling bag is marked with a visual indicator of a correct, predetermined closing position of the sampling bag after mixing.

2. The filter sampling bag of claim 1, wherein the visual indicator is disposed on the sampling bag at a distance from the mouth opening equal to one third of the height of the bag plus or minus 5% of the height of the sampling bag.

3. The filter sampling bag of claim 2, wherein the visual indicator is disposed on the sampling bag at a distance from the mouth opening equal to one third of the height of the sampling bag.

4. The filter sampling bag of claim 1, wherein the filtering partition has a cut-off threshold of approximately 280 microns.

5. The filter sampling bag of claim 1, wherein the visual indicator comprises a distinct line that traverses laterally across at least one of the side walls of the sampling bag.

6. The filter sampling bag of claim 1, wherein the visual indicator comprises two distinct lines comprising a low line and a high line that traverse laterally across at least one of the side walls of the sampling bag.

7. A sampling set for the preparation of samples of solid and/or nutrient and/or vegetable matter to carry out analyses and/or microbiological tests, the sampling set comprising a mixer and a filter sampling bag, wherein the sampling bag is formed of plastic material, wherein the sampling bag has a bottom, side walls, a mouth opening, and a height, wherein the sampling bag is compartmentalized by a filtering partition delimiting a receiving chamber for a sample and a sampling chamber for permitting a taking of samples, and wherein the sampling bag is marked with a visual indicator of a correct, predetermined closing position of the sampling bag after mixing.

8. The sampling set of claim 7, wherein the visual indicator is disposed on the sampling bag at a distance from the mouth opening equal to one third of the height of the bag plus or minus 5% of the height of the sampling bag.

9. The sampling set of claim 8, wherein the visual indicator is disposed on the sampling bag at a distance from the mouth opening equal to one third of the height of the sampling bag.

10. The sampling set of claim 7, wherein the filtering partition has a cut-off threshold of approximately 280 microns.

11. The sampling set of claim 7, wherein the visual indicator comprises a distinct line that traverses laterally across at least one of the side walls of the sampling bag.

12. The sampling set of claim 7, wherein the visual indicator comprises two distinct lines comprising a low line and a high line that traverse laterally across at least one of the side walls of the sampling bag.

13. A sampling kit for preparing samples for carrying out analyzes and/or microbiological tests, the sampling kit comprising a mixer and a filter sampling bag, wherein the sampling bag is formed of plastic material, wherein the sampling bag has a bottom, side walls, a mouth opening, and a height, wherein the sampling bag is compartmentalized by a filtering partition delimiting a receiving chamber for a sample and a sampling chamber for permitting a taking of samples, and wherein the sampling bag is marked with a visual indicator of a correct, predetermined closing position of the sampling bag after mixing.

14. The sampling kit of claim 13, wherein the visual indicator is disposed on the sampling bag at a distance from the mouth opening equal to one third of the height of the bag plus or minus 5% of the height of the sampling bag.

15. The sampling kit of claim 14, wherein the visual indicator is disposed on the sampling bag at a distance from the mouth opening equal to one third of the height of the sampling bag.

16. The sampling kit of claim 13, wherein the filtering partition has a cut-off threshold of approximately 280 microns.

17. The sampling kit of claim 13, wherein the visual indicator comprises a distinct line that traverses laterally across at least one of the side walls of the sampling bag.

18. The sampling kit of claim 13, wherein the visual indicator comprises two distinct lines comprising a low line and a high line that traverse laterally across at least one of the side walls of the sampling bag.

19. A method of manufacturing a filter sampling bag for use in a mixer for the preparation of samples to permit analyses and/or microbiological tests, the method comprising a step of providing a filter sampling bag with a bottom, side walls, a mouth opening, and a height wherein the sampling bag is compartmentalized by a filtering partition delimiting a receiving chamber for a sample and a sampling chamber for permitting a taking of samples and a step of marking the sampling bag with a visual indicator of a correct, predetermined closing position of the sampling bag after mixing.

20. The method of claim 19, further comprising, a step of establishing a closed position of the sampling bag in the mixer and, prior to the marking step, a step of determining the position of the visual indicator from the closed position of the sampling bag in the mixer.

21. The method of claim 19, wherein the visual indicator is disposed on the sampling bag at a distance from the mouth opening equal to one third of the height of the bag plus or minus 5% of the height of the sampling bag.

22. The method of claim 19, wherein the visual indicator comprises a distinct line that traverses laterally across at least one of the side walls of the sampling bag.

23. The method of claim 19, wherein the visual indicator comprises two distinct lines comprising a low line and a high line that traverse laterally across at least one of the side walls of the sampling bag.
